(19) United States
(12) Patent Application Publication
(10) Pub. No.: US 2015/0119943 A1
(43) Pub. Date: Apr. 30, 2015

(54) SYSTEMS AND METHODS FOR SECURING A BONE PLATE

(71) Applicant: Emerge Medical, Inc., Denver, CO (US)

(72) Inventors: Michael J. Milella, JR., Richmond, IL (US); John P. Marotta, Denver, CO (US); Katy Ren'e Swanson, Englewood, CO (US)

(21) Appl. No.: 14/066,271

(22) Filed: Oct. 29, 2013

(51) Int. Cl. A61B 17/17; A61B 17/80

(52) U.S. Cl. CPC A61B 17/1728; A61B 17/8057
USPC 606/281; 606/96; 606/280

(57) ABSTRACT

A drill guide includes an outer body and an inner sleeve. The outer body defines a main bore. The outer body includes a tip extending therefrom, wherein the tip is coaxial with the main bore. The tip includes a plurality of deflectable leaves. The inner sleeve defines a receiving bore, and the inner sleeve is adapted to be removably received within the main bore.

Bone plates are used to fix the position and orientation of portions of broken or fractured bones while the bone heals. To ensure that the plate is properly positioned relative to the bone, the plate is first temporarily oriented and secured with clamps, K-wires, or other implements. The bone is then drilled at one or more locations consistent with the location of one or more openings on the plate. Bores formed by the drill should be aligned with the openings on the plate to ensure proper securement of the plate. To ensure alignment, guides are secured to the plate at each opening in advance of use. The guides dictate the angle at which the drill will be directed into the bone. After drilling, each guide is disconnected from the plate and screws are used to secure the plate to the bone, via the openings.

### SUMMARY

In one aspect, the technology relates to a drill guide including: an outer body defining a main bore, the outer body including a tip extending from the body, wherein the tip is coaxial with the main bore and includes a plurality of deflectable leaves; and an inner sleeve defining a receiving bore, wherein the inner sleeve is adapted to be removably received within the main bore. In another aspect, the technology relates to a bone plate mounting system including: a bone plate defining at least one threaded opening, wherein the at least one threaded opening includes an opening trajectory axis; and a drill guide, wherein the drill guide includes an outer body defining a bore including a bore axis, and wherein the outer body includes a tip extending from the body, and wherein the tip is coaxial with the bore axis and includes a plurality of deflectable leaves, and wherein the leaves are a threaded surface adapted to mate with the at least one threaded opening when the leaves are deflected away from the bore axis. In another aspect, the technology relates to a method of mounting a bone plate to a bone, the method including: placing a bone plate proximate a bone, wherein the bone plate defines a first opening including a first alignment element and a first opening trajectory axis; inserting a tip of a drill guide into the first opening, wherein the tip of the drill guide includes a tip alignment structure; and expanding the tip such that the tip alignment structure mates with the first alignment element.

### BRIEF DESCRIPTION OF THE DRAWINGS

There are shown in the drawings, embodiments which are presently preferred, it being understood, however, that the technology is not limited to the precise arrangements and instrumentalities shown.

FIGS. 1A and 1B depict first and second side views of a drill guide in accordance with one embodiment of the technology.

FIG. 1C depicts a cross-sectional view of the drill guide of FIG. 1A.

FIG. 2 depicts an enlarged cross-sectional view of a tip of a drill guide.

FIGS. 3A-3D depict perspective views of a drill guide in accordance with another embodiment of the technology.

FIG. 4 depicts a bone plate in accordance with one embodiment of the technology.

FIGS. 5A-5C depicts a method of utilizing a drill guide during securement of a bone plate.

FIG. 6 depicts a partial enlarged cross-sectional view of a drill guide secured to a bone plate.

FIG. 7 depicts a method of securing a bone plate to a bone.

### DETAILED DESCRIPTION

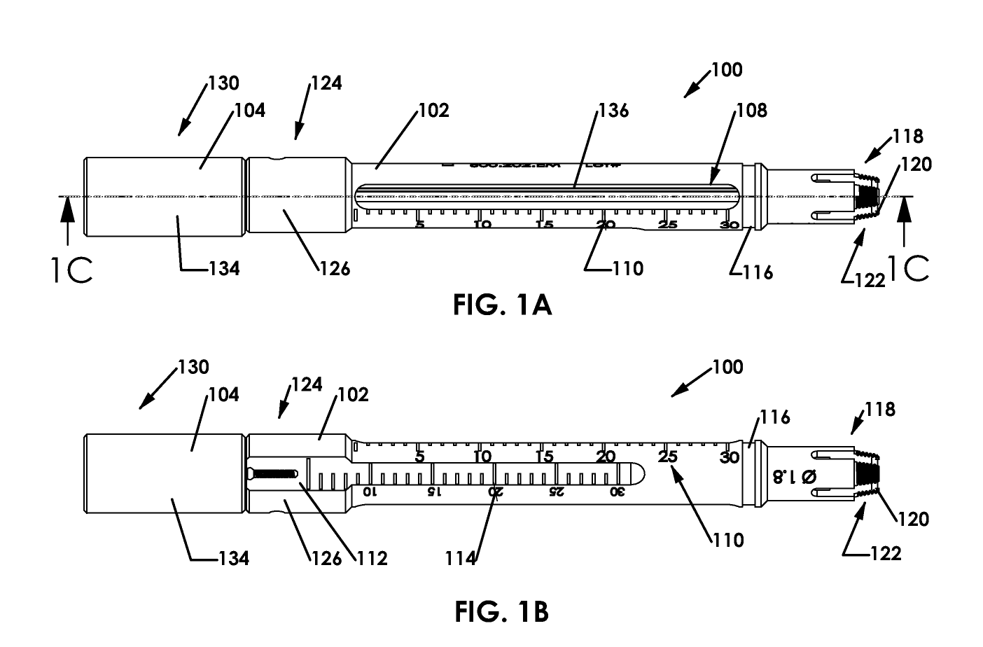

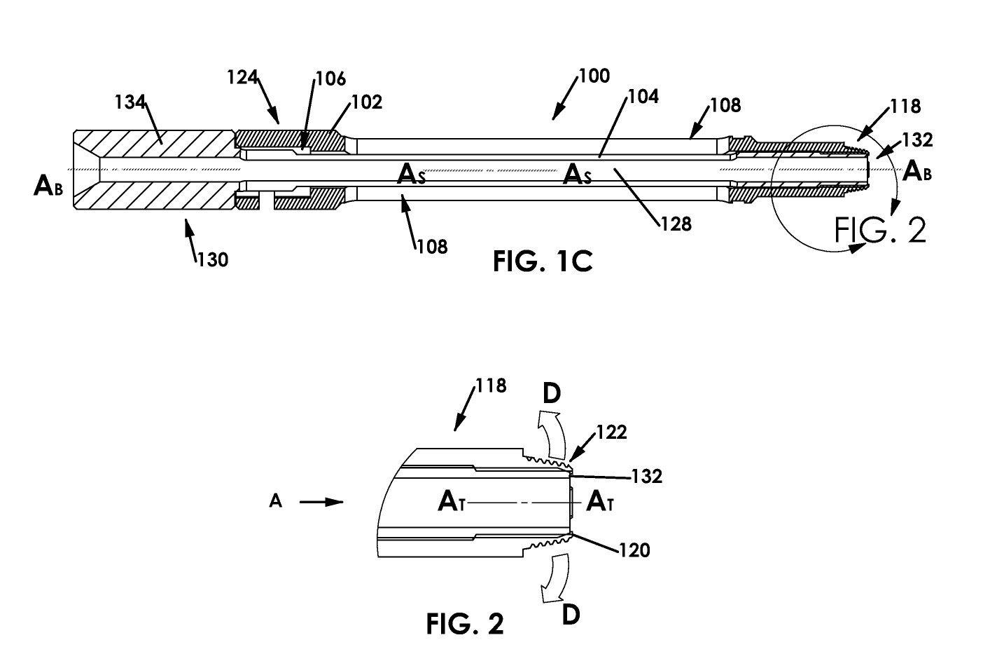

A drill guide 100 for use in securing a bone plate to a bone is depicted in FIGS. 1A-1C, which are described simultaneously. The drill guide 100 includes an outer body 102 and an inner sleeve 104. The outer body 102 defines a main bore 106 into which the inner sleeve 104 is inserted during use. The outer body 102 defines at least one window 108 that may be utilized during operation procedures, as described in more detail below. The window 108 may include indicia 110 proximate thereto that may be used to monitor a drilling procedure, to help ensure a proper depth of drilling is attained. An outer surface of the outer guide 102 may also at least partially define a recess 112. This recess 112 may be used to aid a surgeon in measuring screws used during securement of a bone plate to a bone. Since multiple screws of different lengths are often used to secure a single bone plate to a bone, the recess 112 allows a surgeon or assistant to quickly and accurately identify the size of screws being used in a particular procedure. Indicia 114 indicative of screw length may also be present proximate the recess 112.

The drill guide 100 may also include other references or markings to help identify bore size or other characteristics to a user. For example, an outer ring 116 may be color coded or otherwise marked to identify, e.g., a diameter of the main bore 106 of the outer body 102. This too helps ensure the proper screws are used with the particular drill guide 100. The outer body 102 includes a distal tip 118 that includes a number of deflectable leaves 120. In the depicted embodiment, four deflectable leaves 120 are utilized, but embodiments using a different number of leaves are also contemplated. In general, it may be desirable that an even number of leaves 120 are used, such that deflection of the leaves 120 applies an even pressure to an associated bone plate opening. This deflection process is described in more detail below. The distal tip 118 includes a tip alignment structure 122 that helps secure the distal tip 118 to a mating structure in an opening of a bone plate. The tip alignment structure 122 may comprise one or more projections, elements, detents, surfaces, textures, or other features that are configured so as to mate with a matching structure on openings of a bone plate. In the depicted embodiment, the tip alignment structure 122 is a threaded connection. A threaded connection may be desirable in certain embodiments since the threads may also be used in conjunction with attachment screws. Keys and keyways, pin and recesses, projections and detents, and other structures that ensure mating engagement and alignment are also contemplated. A proximal end 124 of the outer body 102 may include an enlarged base 126, which may be knurled or otherwise textured for ease of gripping and manipulation.

The inner sleeve 104 is received within the main bore 106 during use. The inner sleeve 104 defines a receiving bore 128 sized to receive a bit from a drill. The inner sleeve 104 includes a proximal end 130 and a distal end 132. The proximal end 130 may include an enlarged base 134, which may be knurled or otherwise textured for ease of gripping and manipulation. The distal end 132 is sized slightly larger than an interior diameter of the distal tip 118, which allows the distal end 132 to deflect or otherwise spread the leaves 120 of the distal tip 118 when filling inserted to the outer guide 120. FIG. 2 depicts this condition. As the distal end 132 of the inner sleeve 104 is advanced A within the main bore 106 of the outer guide 102, the distal end 132 contacts the inner portion of the leaves 120, such that the leaves deflect D outward. The leaves 120 are configured such that in their neutral state, they are tapered towards a central axis of the tip 118, and thus return to their neutral state upon removal of the inner sleeve 104 from the main bore 106. In another embodiment, the leaves 120 are straight and spread outward as the distal end 132 contacts the inner surfaces thereof. When the inner sleeve 104 is oriented in the outer body, an elongate slot 136 formed in the inner sleeve 104 is aligned with the window 108.

Each of the outer body 102, the inner sleeve 104, and the tip 118 include an axis A_{B}, A_{S}, and A_{T}, respectively. Since the distal tip 118 extends from the outer body 102, the respective axes A_{T} and A_{B} are generally coaxial. When inserted into the outer body 102, the inner sleeve axis A_{S} is coaxial with both the tip axis A_{T} and the body axis A_{B}. This alignment helps ensure proper orientation of the various components of the drill guide 100, relative to a bone plate.

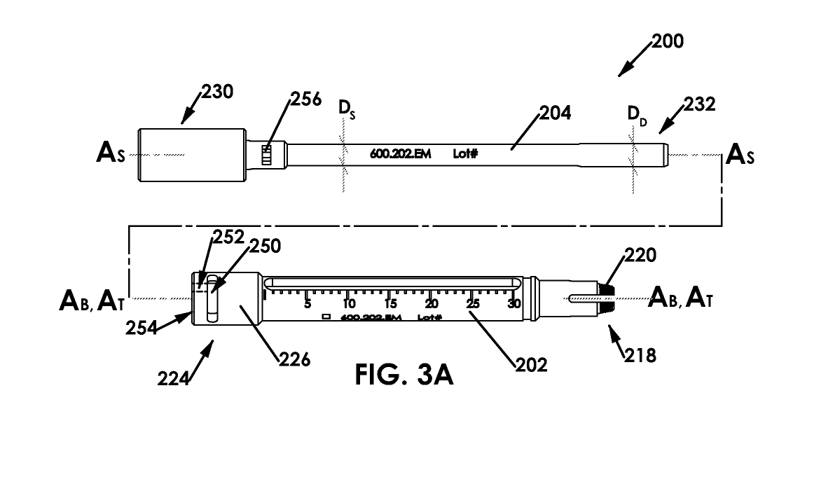

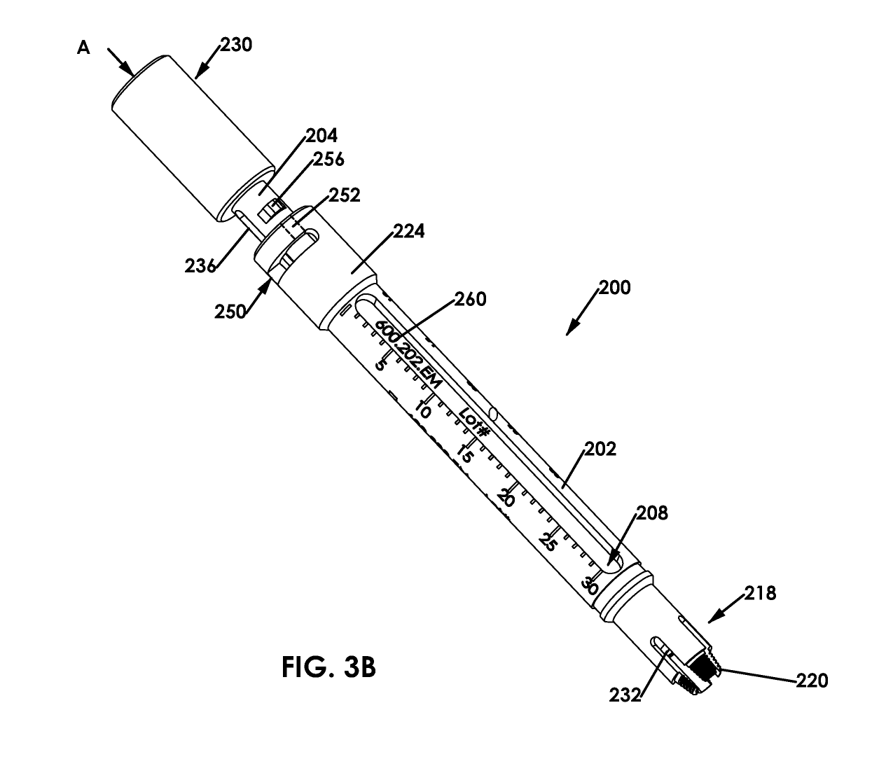

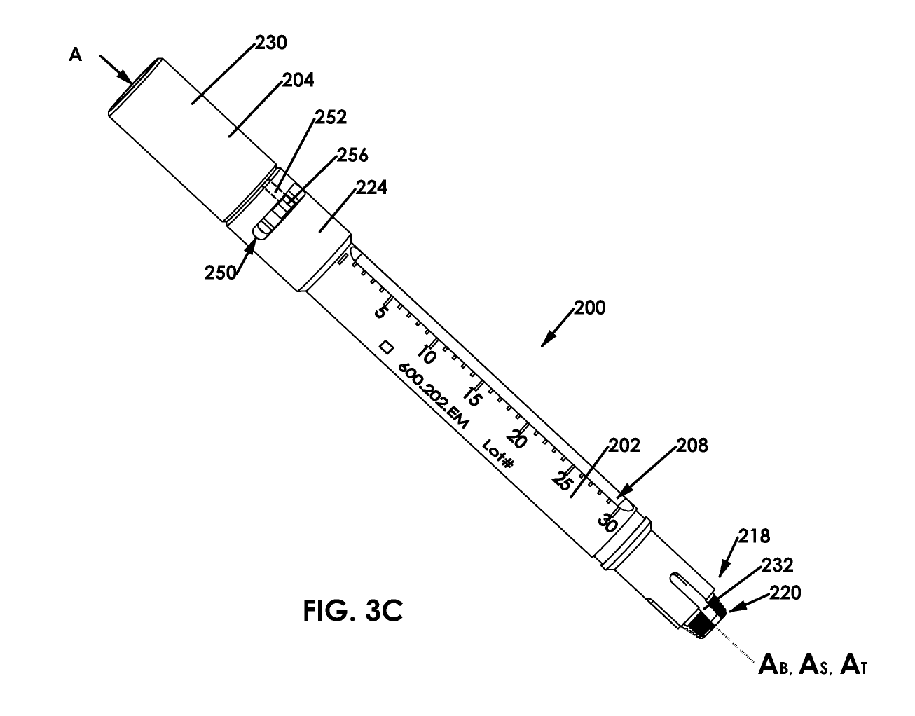

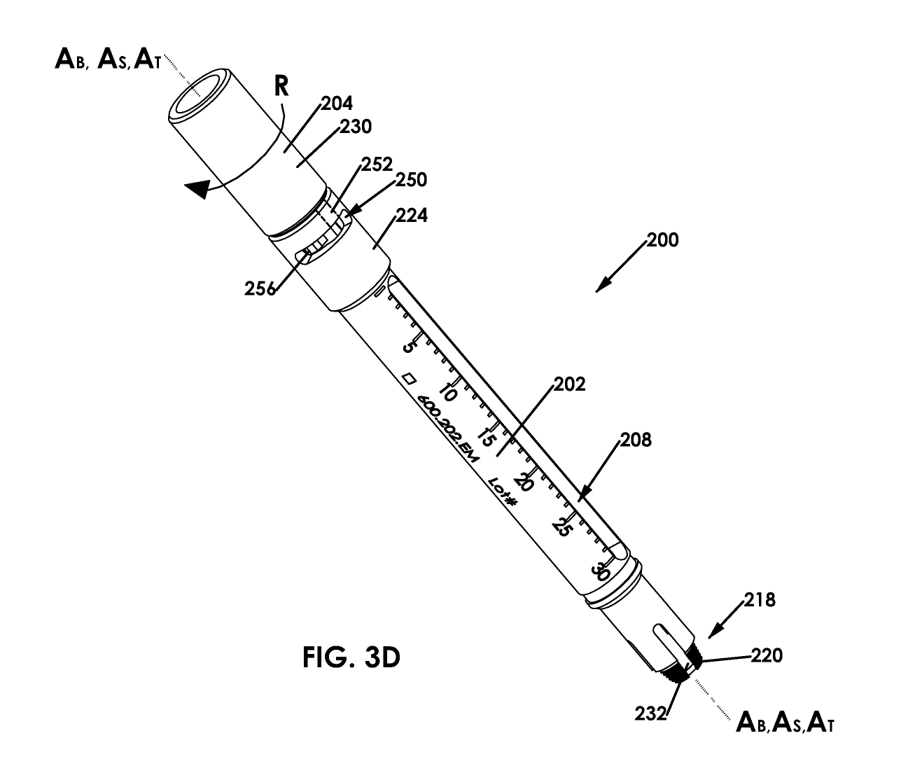

FIGS. 3A-3D depict a drill guide 200 in accordance with another embodiment of the technology. Elements identified by reference numerals similar to those of the drill guide 100 of FIGS. 1A-1C are not described further unless otherwise noted. In FIG. 3A, the inner sleeve 204 is removed from the outer body 202. In this embodiment, a central portion of the inner sleeve 204 has a diameter D_{S} that is less than a diameter D_{D} of the distal tip 232. The enlarged distal tip 232 deflects the leaves 220 away from the body and tip axes A_{B}, A_{T}, respectively, when the inner sleeve 204 is inserted into the main bore of the outer body 202. By utilizing a smaller sleeve diameter D_{S}, friction during insertion of the inner sleeve 204 is reduced, while axial alignment of the body axis A_{B} and the sleeve axis A_{S} is maintained. In other embodiments, the diameter of the inner sleeve 204 may be consistent along its length.

In the depicted embodiment, the proximal end 224 of the outer body 202 may define a slot or channel 250. The slot or channel 250 is connected via a keyway 252 to an outer surface 254 of the enlarged base 226. The keyway 252 is adapted to receive a key, tab, or other projection 256 disposed proximate the enlarged base 234 of the inner sleeve 204. The function of the slot 250 and key 256 are described with regard to FIGS. 3B-3D.

In FIG. 3B, the inner sleeve 204 has been inserted almost completely into the bore of the outer body 202. In this embodiment, the inner sleeve 204 defines an elongate slot 236 that is disposed so as to align with a window 208 disposed on the outer body 202 when the inner sleeve 204 is locked to the outer body 202. As the inner sleeve 204 is advanced A into the outer body 202, indicia or other markings 260 as the inner sleeve 204 may be viewed through the window 208. This aids the user in aligning the key 256 with the keyway 252, which may be difficult to view due to the small size and/or arrangement of the key 256 and keyway 252. As can be seen, in the position depicted in FIG. 3B, the distal tip 232 has not yet advanced far enough to deflect the leaves 220 at the distal tip 218 of the outer body 202. As the inner sleeve 204 is advanced A further, the key 256 passes through the keyway 252 and reaches the slot 250. At this position, depicted in FIG. 3C, the distal tip 232 has advanced far enough to deflect the leaves 220 outward, away from the axes A_{B}, A_{S}, A_{T}, which are now coaxial. In FIG. 3D, the inner sleeve 204 is rotated R typically by gripping and turning the enlarged proximal end 230. This fixes the inner sleeve 204 within the outer body 202, due to the key 256 being held in place in the slot 250. When in the position of FIG. 3D, the elongate slot 236 on the inner sleeve 204 is aligned with the window 208 on the outer body 202.

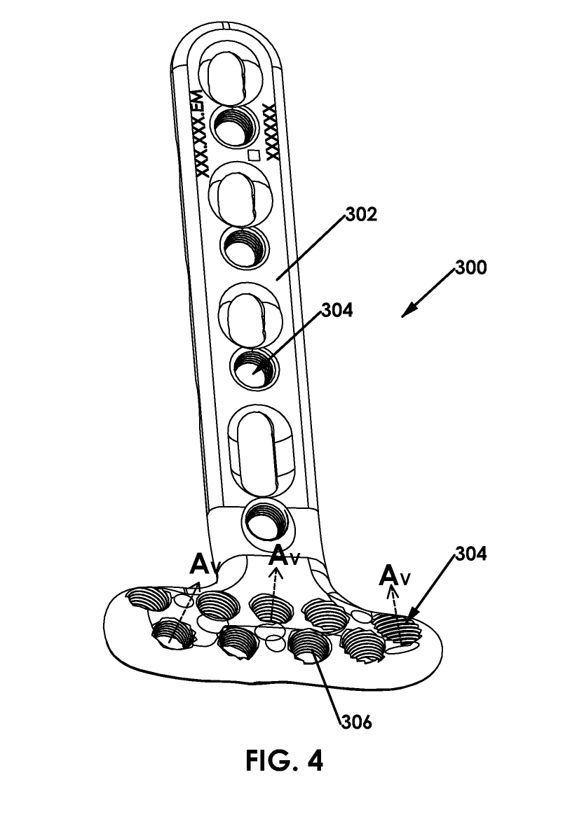

FIG. 4 depicts a bone plate 300, specifically, a distal radius plate, which may be used to set a fracture of the forearm of a human patient. Other plates for other types of bones, fractures, etc., may be utilized with the drill guides described herein. The plate 300 includes a body that defines a number of openings 304 for receiving screws that are used to secure the plate 300 to a bone. Each of the openings 304 includes a mating structure 306 that helps secure the tip of a drill guide thereto. As described above, the tip includes a tip aligning structure that mates with the mating structure 306 in the opening 304 of the bone plate 300. As discussed above, the tip alignment structure may comprise one or more projections, elements, detents, surfaces, textures, or other features that are configured so as to mate with the mating structure 306 on the openings 304. In the depicted embodiment, the mating structure 306 is a threaded connection which may be particularly desirable for the reasons described above. Keys and keyways, pin and recesses, projections and detents, and other structures that ensure mating engagement and alignment are also contemplated.

Each opening 304 defines a trajectory axis A_{V}, which is the axis along which a screw inserted into the opening 304 advances. As can be seen in FIG. 4, these trajectory axes A_{V} can be unique for each opening 304, although some openings share a similar or substantially similar trajectory axis A_{V}. To prevent damage to the underlying bone and assure an approach angle of the screw such that it properly mates with the opening 304, the underlying bone should be pre-drilled along the trajectory axis A_{V}. This process is described below.

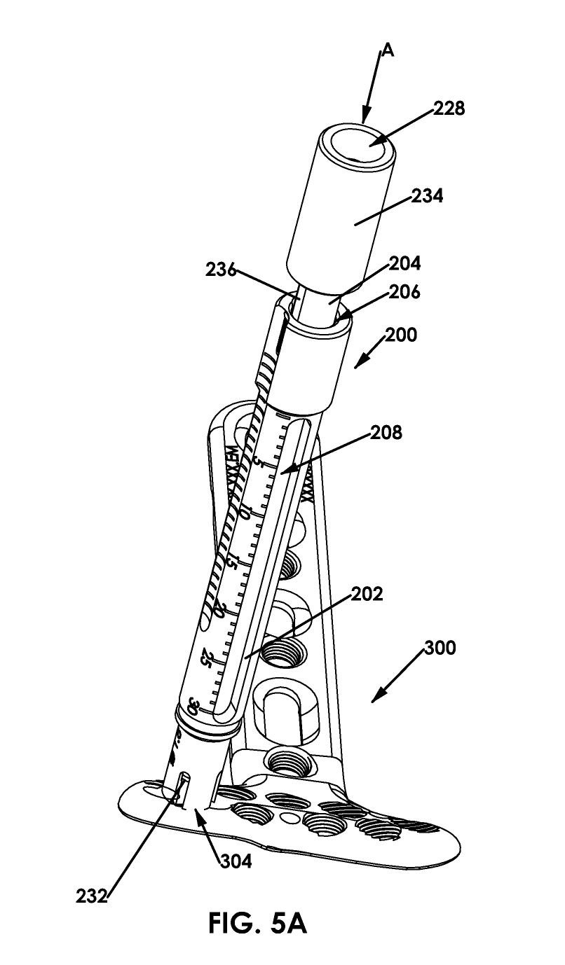

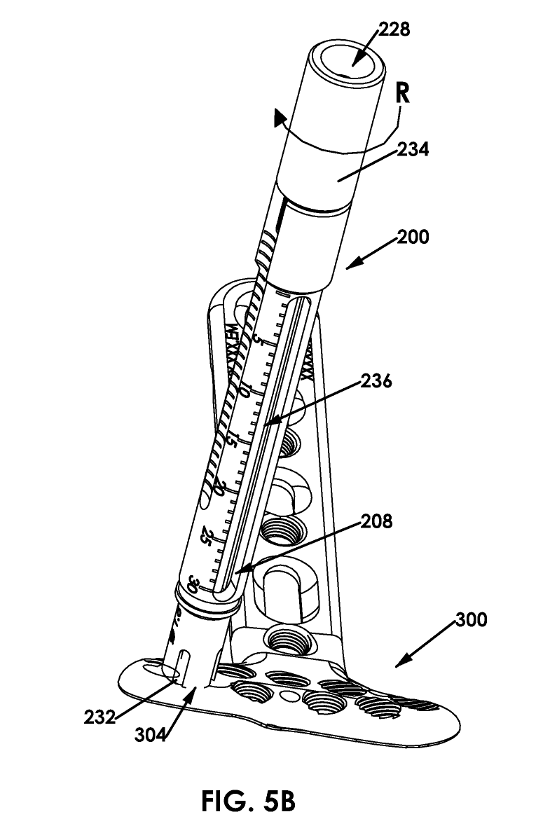

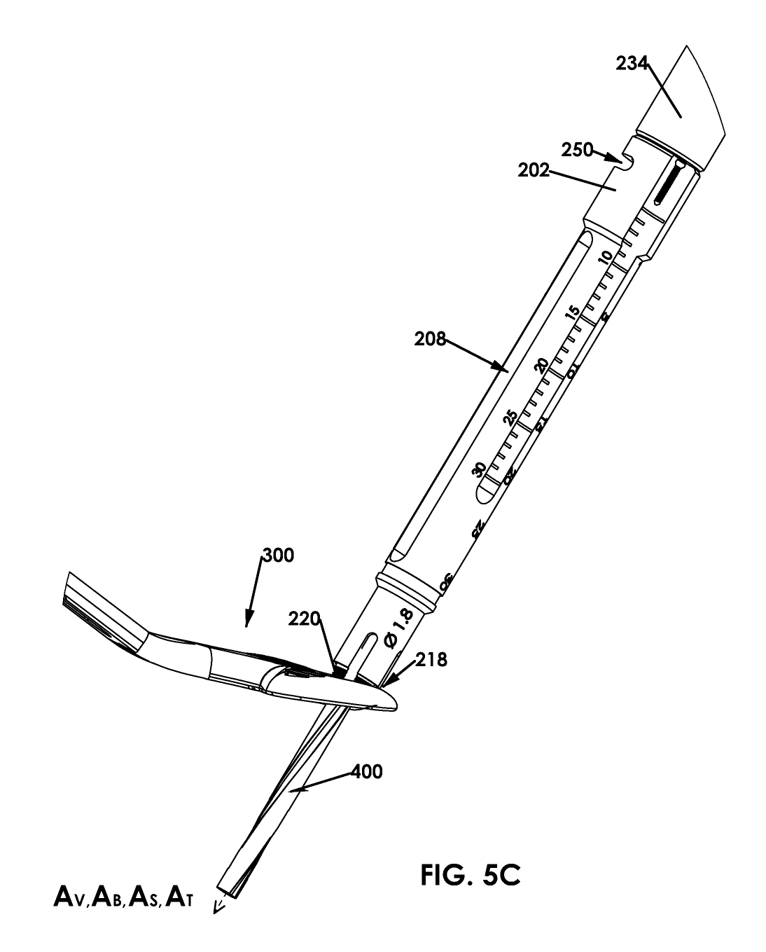

FIGS. 5A-5C depict a method of using a drill guide 200 to drill a bone in preparation for securement of a bone plate 300 thereto. In FIG. 5A, the distal tip (hidden in this figure) of the outer body 202 is inserted into an opening 304 on the bone plate 300. Although the mating structures on the distal tip and the opening 304 may partially mate, the distal tip is not secured until the sleeve 204 is fully advanced A into the outer body 202. In FIG. 5A, the distal tip 232 of the inner sleeve 204 has not been fully advanced into the outer body 202. In FIG. 5B, the distal tip 232 has been completely advanced into the outer body 202, thus locking the position of the drill guide 200 elongate to the bone plate 300. The inner sleeve 204 may be rotated R as described above so as to lock the inner sleeve 204 in position. In the locked position, the elongate slot 258 is aligned with the window 208 so as to allow the receiving bone 236 of the inner sleeve 204 to be visible to the user.

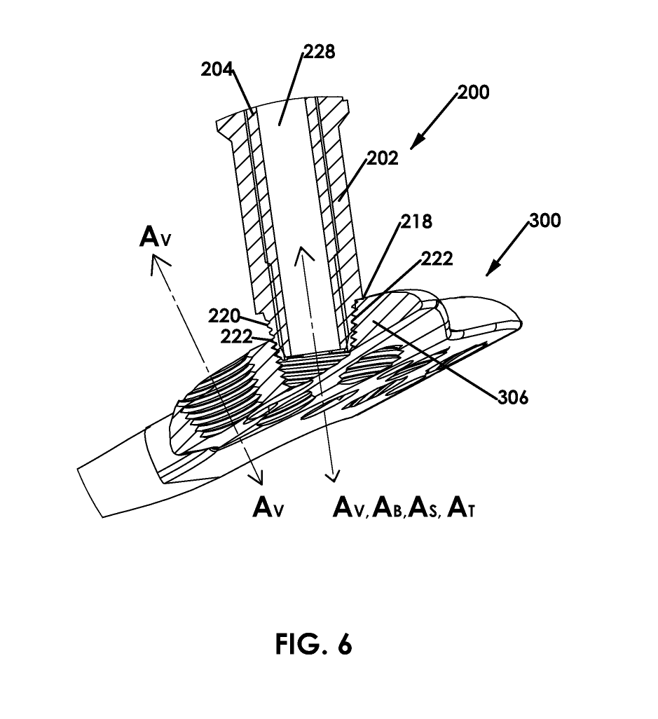

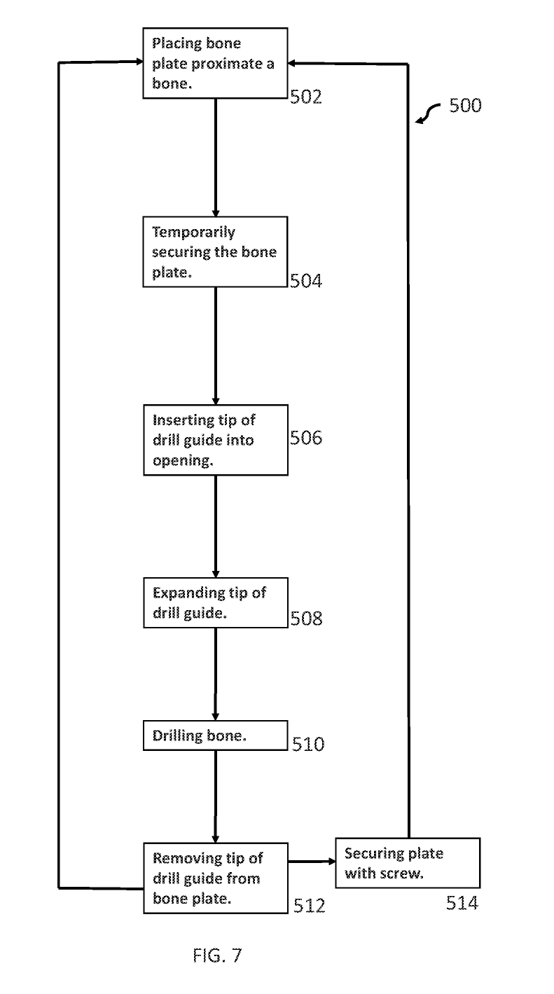

Fully advancing the inner sleeve 204 into the outer body 202 engages the mating alignment structures 222, 306 on the tip 218 and the opening 304, respectively, as depicted in FIG. 6. This mating secures the drill guide 200 relative to the plate 300 and also aligns all of the axes A_{V}, A_{B}, A_{S}, and A_{T}. With these axes aligned, the drill guide 200 is oriented coaxial to the trajectory axis A_{V} of the opening 304, thus ensuring the proper approach of a drill bit to form an opening in the bone. As depicted in FIG. 6, the tip 218 does not extend below a bottom surface of the bone plate 300. This prevents a downward force on the tip 218 from applying pressure to the underlying bone, which could potentially displace the plate 300 relative to the bone. In certain embodiments, however, it may be advantageous to extend the tip 218 through the opening 306.

The pre-drilling operation is depicted in FIG. 5C. A drill bit 400 is inserted via the receiving bore 228. Due to the co-axial alignment of the four axes A_{V}, A_{B}, A_{S}, and A_{T}, the angle of the bit 400 is assured as it drills into the bone. The depth of penetration of the drill bit can be determined by viewing a position of the drill bit via the window 208 and elongate slot 236. Thus, the surgeon can be assured that the bit 400 has been advanced to the correct depth during the procedure.

FIG. 7 depicts a method 500 of mounting a bone plate to a bone. The method 500 begins by placing a bone plate proximate a bone in operation 502. The bone plate is then temporarily secured in operation 504 with K-wires, clamps, or other easily removable fasteners. The tip of a drill guide is inserted into one of the openings on the bone plate in operation 506. In bone plates having multiple openings, the surgeon may decide in which opening to insert the tip based on particular requirements of the surgery, personal experience, or other factors. The tip of the guide is then expanded in operation 508. As described above, the tip of the guide may be expanded by inserting an inner sleeve into a main bore of the outer body. Once the position of the drill guide is fixed due to expansion of the tip, a drill bit may be inserted into a receiving bore of the inner sleeve and the drill activated. The drill is advanced as required or desired for a particular application to drill the bone in operation 510. Once the desired drill opening is complete, the bit may be withdrawn from the inner sleeve, then the inner sleeve withdrawn from the outer body. This enables the tip to be easily removed from the bone plate in operation 512. Thereafter, the surgeon may secure the plate with a screw in operation 514 before drilling at additional openings. In an alternative embodiment, the surgeon may pre-drill multiple holes in the bone (i.e., repeat the method 500) before securing the plate.

Materials utilized in the manufacture of the drill guide may be those typically used in surgical equipment. Stainless steel, titanium, and other robust metals that may be sterilized may be used. Aluminum, anodized aluminum, and rigid polymers also may be utilized. Carbon fiber-reinforced polymers may be utilized, as they are lightweight, extremely strong, and may be sterilized. Of course, drill guides utilizing a combination of materials may be used.

The drill guides and plates depicted herein may be sold as a kit including the components necessary for one-time use of a drill guide to secure a plate to a bone. In certain embodiments, the kit may include a plate, a drill guide having an outer guide and one or more inner sleeves, and a plurality of screws. Multiple sleeves having different receiving bore diameters may be included in the kit so a variety of different size screws may be used. In other embodiments, a single drill guide with a single inner sleeve and the appropriately-sized drill bit and screws may be included in the kit. Although the outer body and inner sleeve of the drill guide may be re-used, it may be desirable for the drill guide to be packaged as a single-use device, with instructions for disposal after use thereof.

While there have been described herein what are to be considered exemplary and preferred embodiments of the present technology, other modifications of the technology will become apparent to those skilled in the art from the teachings herein. The particular methods of manufacture and geometries disclosed herein are exemplary in nature and are not to be considered limiting. It is therefore desired to be secured in the appended claims all such modifications as fall within the spirit and scope of the technology. Accordingly, what is desired to be secured by Letters Patent is the technology as defined and differentiated in the following claims, and all equivalents.

What is claimed is:

1. A drill guide comprising:
an outer body defining a main bore, the outer body comprising a tip extending from the body, wherein the tip is coaxial with the main bore and comprises a plurality of deflectable leaves; and
an inner sleeve defining a receiving bore, wherein the inner sleeve is adapted to be removably received within the main bore.

2. The drill guide of claim 1, wherein the inner sleeve deflects the plurality of deflectable leaves when inserted into the sleeve.

3. The drill guide of claim 1, wherein at least one of the plurality of leaves comprises a set of threads.

4. The drill guide of claim 1, wherein the plurality of leaves are tapered toward an axis of the main bore.

5. The drill guide of claim 1, wherein at least one of the plurality of leaves comprise an alignment element, wherein the alignment element is adapted to mate with a mating element of a hole defined by a bone plate.

6. The drill guide of claim 5, wherein the receiving bore comprises a bore axis, and wherein engagement of the alignment element and the mating element aligns the bore axis with a hole trajectory axis of the hole defined by the bone plate.

7. The grill guide of claim 5, wherein the alignment element and the mating element comprise mating threaded contours.

8. A bone plate mounting system comprising:
a bone plate defining at least one threaded opening, wherein the at least one threaded opening comprises an opening trajectory axis; and
a drill guide, wherein the drill guide comprises an outer body defining a bore comprising a bore axis, and wherein the outer body comprises a tip extending from the body, and wherein the tip is coaxial with the bore axis and comprises a plurality of deflectable leaves, and wherein the leaves comprise a threaded surface adapted to mate with the at least one threaded opening when the leaves are deflected away from the bore axis.

9. The bone plate mounting system of claim 8, wherein the drill guide further comprises an inner sleeve defining a receiving bore, wherein the inner sleeve is adapted to be removably received within the bore.

10. The bone plate mounting system of claim 9, wherein when the inner sleeve is received within the bore, the inner sleeve deflects the leaves away from the bore axis.

11. The bone plate mounting system of claim 9, wherein when the inner sleeve is received within the bore, the receiving bore is coaxial with the bore axis.

12. The bone plate mounting system of claim 10, wherein the leaves are tapered towards the bore axis.

13. The bone plate mounting system of claim 10, wherein the leaves are biased towards the bore axis when the inner sleeve is received within the bore.

14. The bone plate mounting system of claim 9, wherein the inner sleeve comprises a lock for securing the inner sleeve to the outer body.

15. A method of mounting a bone plate to a bone, the method comprising:
placing a bone plate proximate a bone, wherein the bone plate defines a first opening comprising a first alignment element and a first opening trajectory axis;
inserting a tip of a drill guide into the first opening, wherein the tip of the drill guide comprises a tip alignment structure; and
expanding the tip such that the tip alignment structure mates with the first alignment element.

16. The method of claim 15, wherein the tip comprises a tip axis, wherein when the tip alignment structure mates with the first alignment element, the tip axis is coaxial with the first opening trajectory axis.

17. The method of claim 15, wherein the expanding operation comprises inserting an inner sleeve into a bore defined by the drill guide.

18. The method of claim 15, wherein the first alignment element and the tip alignment structure comprise mating threaded surfaces.

19. The method of claim 15, further comprising:
removing the tip from the first opening;
inserting the tip into a second opening defined by the plate, wherein the second opening comprises a second alignment element and a second opening trajectory axis disposed skew to the first opening trajectory axis; and
expanding the tip such that the tip alignment structure mates with the second alignment element.

20. The method of claim 17, further comprising inserting a drill bit through a bore in the inner sleeve.
